(19) United States
(12) Patent Application Publication
(10) Pub. No.: US 2005/0222031 A1
(43) Pub. Date: Oct. 6, 2005

(54) Method for preventing or treating malignant melanoma

(76) Inventors: Sho-ichi Yamagishi, Fukuoka-ken (JP); Tsutomu Imaizumi, Fukuoka-ken (JP); Hiroshi Shimizu, Hokkaido (JP); Riichiro Abe, Hokkaido (JP)

Correspondence Address: WENDEROTH, LIND & PONACK, L.L.P., 2033 K STREET N. W., SUITE 800, WASHINGTON, DC, 20006-1021, US

(21) Appl. No.: 11/092,721

(22) Filed: Mar. 30, 2005

(30) Foreign Application Priority Data
Mar. 19, 2004 (JP) 2004-80387

(51) Int. Cl. A61K 38/18

(52) U.S. Cl. 514/12

(57) ABSTRACT

Provided is a novel method for preventing or treating malignant melanoma, which comprises administering an effective amount of at least one selected from the group consisting of: a) a pigment epithelium-derived factor; b) a variant of the pigment epithelium-derived factor (a) that has the functionally equivalent properties to the factor (a); and c) a vector comprising such factor to a subject in need thereof.

### TECHNICAL FIELD

The invention relates to a method for preventing or treating malignant melanoma.

### BACKGROUND OF THE INVENTION

A major event in tumor growth and expansion is the “angiogenic switch”, an alteration in the balance of pro-angiogenic and anti-angiogenic molecules that leads to tumor neovascularization (1). Many tumors not only overexpress multiple angiogenic factors such as human vascular endothelial growth factor (VEGF), basic fibroblast growth factor and interleukin-8, but also underexpress angiogenic inhibitors such as thrombospondin-1, thus favoring angiogenesis (11, 12).

Angiogenesis, a process by which new vascular networks are formed from pre-existing capillaries, is required for tumors to grow, invade and metastasize (1, 2). Tumors are unable to grow beyond a volume of 1-2 mm³ without establishing a vascular supply because cells must be within 100-200 μm of a blood vessel to survive (1, 2). Tumor vessels are genetically stable, and less likely to accumulate mutations that allow them to develop drug resistance in a rapid manner (3). Therefore, targeting vasculatures that support tumor growth, rather than cancer cells, is considered one of the most promising approaches to cancer therapy.

Pigment epithelium-derived factor (PEDF), a glycoprotein that belongs to the superfamily of serine protease inhibitors, was first purified from human retinal pigment epithelial cell conditioned media as a factor with potent human retinoblastoma cell neuronal differentiating activity (4). Recently, PEDF has been shown to be a potent inhibitor of angiogenesis in both cell culture and animal models. Indeed, PEDF is reported to inhibit retinal endothelial cell growth, migration and suppress ischemia-induced retinal neovascularization (5, 6). Furthermore, loss of PEDF was associated with angiogenic activity in proliferative diabetic retinopathy (7). PEDF is also known to effectively suppress retinal and choroidal neovascularization caused by ischemia and age-related macular degeneration, respectively (6, 14). very recently, Doll et al reported that PEDF-deficient mice likely suffer from pancreas cancers (15). Anyway, a functional role for PEDF in tumor growth and angiogenesis remains to be elucidated.

Further, PEDF has been described to up-regulate Fas ligand in endothelial cells, thereby specifically sensitizing tumor vessels to apoptosis (16).

In this study, we investigated both in vitro and in vivo growth characteristics of the human malignant melanoma cell line G361, stably transfected to overexpress human PEDF.

Pigment epithelium-derived factor (PEDF) has recently been shown to be the most potent inhibitor of angiogenesis in the mammalian eye, and is involved in the pathogenesis of angiogenic eye disease such as proliferative diabetic retinopathy.

### SUMMARY OF THE INVENTION

In this invention, we have investigated both the in vitro and in vivo growth characteristics of human malignant melanoma G361cell lines, stably transfected to overexpress human PEDF. Expression levels of PEDF proteins in melanoma cell lines G361 and A375 were comparable with that of human cultured melanocytes, while vascular endothelial growth factor levels in those two tumor cell lines were much stronger than that in normal melanocytes. Overexpression of PEDF was found to significantly inhibit tumor growth and vessel formation in G361 nude mice xenografts. Furthermore, in vitro proliferation rates of G361 cells were decreased in PEDF-transfected cells. PEDF proteins showed dose-dependent induced growth retardation and apoptotic cell death in non-transfected G361 cells, which were completely prevented by treatment with antibodies against the Fas ligand. The present invention highlights two beneficial effects of PEDF treatment on growth and expansion of malignant melanoma; one is the suppression of melanoma angiogenesis, and the other is induction of Fas ligand-dependent apoptosis in melanoma cells, not tumor vessels. PEDF therefore might be a promising novel therapeutic agent for prevention and/or treatment of patients with malignant melanoma.

Accordingly, in one aspect of the invention, a method for preventing or treating malignant melanoma, which comprises administering at least one selected from the group consisting of:
a) a pigment epithelium-derived factor; and
b) a variant of the pigment epithelium-derived factor (a) that has the functionally equivalent properties to the factor (a) to a subject in need thereof, is provided.

In another aspect of the invention, a method for preventing or treating malignant melanoma, which comprises administering a vector that comprises the nucleic acid molecule encoding at least one selected from the group consisting of:
a) a pigment epithelium-derived factor; and
b) a variant of the pigment epithelium-derived factor (a) that has the functionally equivalent properties to the factor (a) to a subject in need thereof, is also provided.

### BRIEF DESCRIPTION OF THE DRAWINGS

FIG. 1 shows immunofluorescence staining (A) and western blot analysis (B) of G361, A375 and normal melanocytes. In FIG. 1A, the insert indicates negative staining without primary Ab. Scale bars, 10 μm.

FIG. 2 shows the results of western blot analysis of conditioned medium from G361 cells, stably transfected with the human PEDF expression vector (lane 2) or with the expression vector alone (lane 3). Purified PEDF proteins (0.2 μg) were used as a positive control (lane 1).

FIG. 3 is a graph showing the effect of PEDF overexpression on growth of melanoma cells in mice transplanted with G361 xenografts. *, P<0.0001 compared with control cells.

FIG. 4 shows tumor vessels in G361 xenografts. Typical photomicrographs of immunofluorescent staining for CD31 (open area) and nuclei as stained with propidium iodide (dashed points) in G361 xenografts. FIG. 4A shows the photomicrograph of G361 cells stably transfected with the human PEDF expression vector. FIG. 4B shows the photomicrograph of G361 cells stably transfected with the expression vector pBK-CMV alone. Scale bar, 50 μm. FIG. 4C shows the quantitative analysis of the fluorescent-positive area (per 1000 μm²) in tumors. *, P<0.0001 compared to control cells.

FIG. 5 is graphs showing growth and apoptosis of G361 cells in vitro. FIG. 5A shows growth rates of G361 cells, stably transfected with the human PEDF expression vector or with the expression vector pBK-CMV alone in vitro. FIGS. 5B and 5C show the effects of PEDF protein on growth and apoptosis of non-transfected G361 cells, respectively. *, P<0.005 compared to the value without treatments.

FIG. 6 shows apoptotic cells in G361 xenografts. Typical photomicrographs of apoptotic cells using TUNEL assay (open area) in G361 xenografts, and nuclei as stained with propidium iodide (dashed points). FIG. 6A shows the photomicrograph of G361 cells, stably transfected with the human PEDF expression vector. FIG. 6B shows the photomicrograph of G361 cells stably transfected with the expression vector pBK-CMV alone. Scale bars, 50 μm. FIG. 6C shows the quantitative analysis of the apoptotic cell percentage in tumors. *, P<0.005 compared to control cells.

### DETAILED DESCRIPTION OF THE PREFERRED EMBODIMENTS

The inventors of the present application have been the first to identify a PEDF-induced growth retardation and induction of apoptotic cell death in melanoma cells that is dependent on the Fas ligand. The invention of the present application is based on the findings that PEDF could directly elicit apoptosis in melanoma cells by inducing Fas ligand on these tumor cells. Recently, granulocytes have been shown to inhibit melanoma lung metastasis by inducing Fas-ligand-associated apoptosis, further supporting our findings (17).

Apoptosis means a process of programmed cell death in which a cell responds to an external stimulus by committing suicide in a highly stereotyped manner. Events in apoptosis include cell shrinkage, degradation of the cell's chromatin and destruction of its genome, and the like. Apoptosis is one of the three general types of behavior of cells (the other 2 types are replication and differentiation), and it plays an indispensable role in development, tissue homeostasis, and response to injury. Apoptosis refer to cell program death that differs from necrosis.

PEDF and a variant thereof can induce apoptosis in melanoma and therefore, may be used to cope with malignant melanoma. In addition, a vector comprising a nucleotide encoding PEDF or a variant thereof can be used to induce apoptosis in melanoma and therefore, may also be used to cope with malignant melanoma.

1. Proteins

The amino acid sequence and the nucleic acid sequence of Pigment epithelium-derived factor (PEDF) are described in Proc. Natl. Acad. Sci. U.S.A. 90 (4), 1526-1530 (1993), and are registered under Genbank Accession No. M76979. For the reference, the amino acid sequence and the nucleic acid sequence of PEDF are shown in SEQ ID Nos.: 1 and 2, respectively. According to the invention, PEDF includes all kinds of PEDF derived from mammals such as human, dog, cat, cow, and horse, with PEDF from human being preferred.

Production of PEDF by expressing the DNA encoding the protein may be achieved in accordance with many publications and references such as “Molecular Cloning”, 2nd ed., Cold Spring Harbor Laboratory Press (1989). Particularly, an expression plasmid is constructed by inserting a DNA of the present invention into an appropriate expression vector (e.g., pBK-CMV). Subsequently, the expression plasmid is introduced into appropriate host cells to obtain transformants. Examples of host cells include those cells of prokaryotes such as Escherichia coli, unicellular eukaryotes such as yeast, and multicellular eukaryotes such as insects or animals. Transfer of expression plasmid into host cells may be achieved by conventional methods such as calcium phosphate method, electric pulse method, Lipofectin method, or the like. Desired proteins are produced by culturing the transformants in appropriate medium according to usual methods. The protein thus obtained may be isolated and purified according to standard biochemical procedures.

As used herein, “variant of PEDF that has the functionally equivalent properties to PEDF” include all kinds of PEDF variants as long as the variants have the functionally equivalent properties to human PEDF. For example, the variants of PEDF are described in, for example, U.S. Pat. No. 6,319,687, WO03059248 and WO9324529.

Preferable examples of the PEDF variants include variants of PEDF that comprise an amino acid sequence that contains alteration of one or more, or several amino acid residues in the amino acid sequence of human PEDF wherein the alteration is substitution, deletion and/or addition, and have the functionally equivalent properties to human PEDF.

Functionally equivalent properties to human PEDF mean suppression of melanoma angiogenesis, and/or induction of Fas ligand-dependent apoptosis in melanoma cells, which are equivalent to that of human PEDF.

The variants according to the invention may also be prepared by recombinant technology as shown above.

It can be demonstrated whether or not a variant prepared as shown above has functionally equivalent properties according to the examples hereinafter.

According to the present invention, a pharmaceutical composition comprising a PEDF or a variant thereof of the present invention and, if necessary, a conventional carrier may be used for the administration.

According to the present invention, a PEDF or a variant thereof may be administered in such a manner that they contact with melanoma cells, for example, intradermally, hypodermically, or by intravenous injection. Preferably, a PEDF or a variant thereof is administered by injection to the site where the melanoma masses exist directly, or by use of an antibody directed to the malignant melanoma. Any conventional method may be used to target melanoma. Although the amount of the protein to be administered may vary depending on the severity of the condition be treated, the age and the weight of the patient, and the like, it is typical to administer 0.0001 mg-1000 mg, preferably 0.001 mg-100 mg, more preferably 0.01 mg-10 mg of a PEDF or a variant thereof every several days to every several months.

2. Nucleic Acids and Vectors

The present invention also provides a method for preventing or treating malignant melanoma which comprising administering a vector that comprises a nucleic acid that encodes a pigment epithelium-derived factor, or a variant thereof that comprises an amino acid sequence that contains alteration of one or several amino acid residues in the amino acid sequence of the pigment epithelium-derived factor wherein the alteration is substitution, deletion and/or addition, and have the functionally equivalent properties to the pigment epithelium-derived factor.

As used herein, “nucleic acid” includes a DNA and an RNA, which may be single-stranded or double-stranded. Nucleic acid can be easily prepared according to typical DNA synthesis or genetic engineering method, for example, according to the description of a standard text such as “Molecular Cloning”, 2nd ed., Cold Spring Harbor Laboratory Press (1989).

According to the present invention, the vector should be designed so that the nucleic acid encoding a PEDF or a variant thereof incorporated in the vector can be expressed in the melanoma cells.

Such vectors mean those useful for gene therapy as used conventionally. Examples of the vectors include viral vectors wherein the nucleic acid as shown above is incorporated into DNA or RNA viruses such as retrovirus, adenovirus, adeno-associated virus, herpesvirus, vaccinia virus, poxvirus, poliovirus, or Sindbis virus, and introduced into cells. Among these methods, those using retrovirus, adenovirus, adeno-associated virus, or vaccinia virus are especially preferred. Specific examples of the vectors include pAd/CMV/V5-DEST Gateway Vector (Invitrogen).

According to the present invention, a pharmaceutical composition comprising such a vector of the present invention as an active ingredient and, if necessary, conventional carriers may be used.

According to the present invention, the vector may be administered, for example, intradermally, hypodermically, or by intravenous injection. Preferably, the vector is administered by injections to the site where the melanoma masses exist directly, or by use of antibodies directed to the melanoma. Any conventional method may be used to target melanoma. Although the amount of the vector to be administered may vary depending on the severity of the condition to be treated, the age and the weight of the patient and the like, it is typical to administer 0.0001 mg-100 mg, preferably 0.001 mg-10 mg, of a nucleic acid every several days to every several months.

### EXAMPLES

The present invention is further illustrated by the following examples, but is not limited by these examples in any respect. All values were presented as means ±S. E. M. (standard error of the mean). Statistical significance was evaluated using the Student's t test for paired comparison; p<0.05 was considered significant.

Reference 1

PEDF and VEGF expression by melanoma cells and normal melanocytes

1.1 Cells

Two human malignant melanoma cell lines G361 and A375 (ATCC Nos. CRL-1424 and CRL-1619, American Type Culture Collection, Manassas, Va.) were maintained in RPMI 1640 supplemented with 10% fetal calf serum (FCS), 100 units/ml penicillin/streptomycin. Normal human neonatal melanocytes were purchased from Kurabo (Osaka, Japan) and maintained according to the manufacturer's instructions.

1.2 Preparation of Polyclonal Antibodies (Ab) Against Human PEDF

Polyclonal Ab against 44-mer PEDF polypeptides (VLLSPLSVATALSALSLGAEQRTESIIHRALYYDLISSFDIHGT: SEQ ID NO: 3) was prepared as previously described (8). We confirmed that the polyclonal Ab actually bound to purified PEDF protein (data not shown).

1.3 Immunofluorescence Microscopy

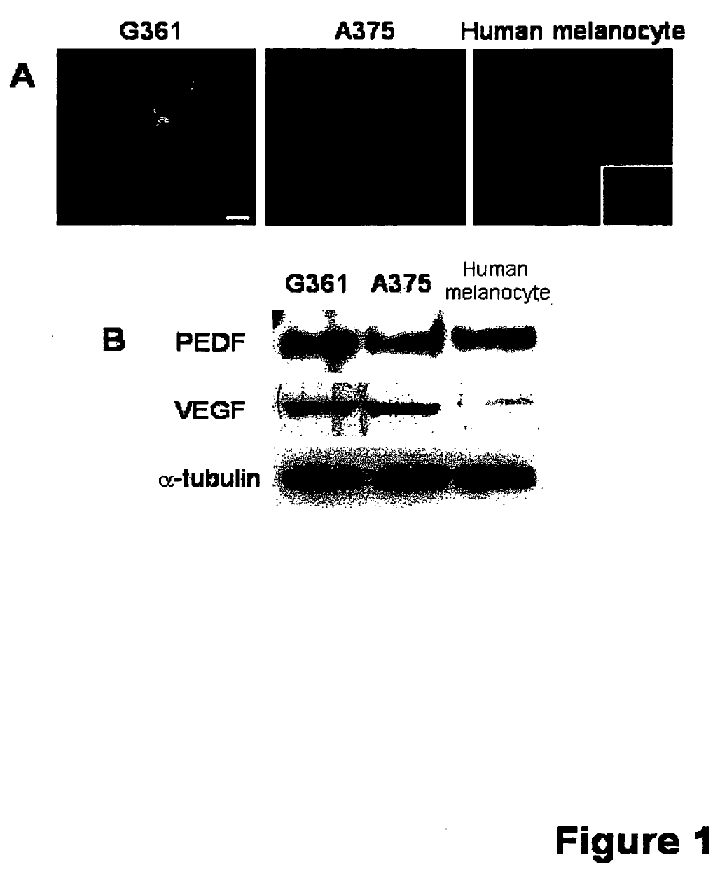

Immunofluorescence staining was performed on G361, A375 or normal melanocytes cultured on glass coverslips. Each cell type was incubated with an anti-PEDF Ab as prepared in above 1.2 at 4° C. overnight, and then these primary Abs were detected with fluorescein isothiocyanate (FITC)-labeled goat anti-rabbit IgG (Vector Laboratories, Burlingame, Calif.). Fluorescence staining was detected using a confocal laser scanning fluorescence microscope (Laser Scanning Confocal Imaging System MRC 1024; Bio-Rad, Richmond, Calif.). The result is shown in FIG. 1A.

1.4 Western Blot Analysis

G361, A375, and normal melanocytes were grown to confluence in 100-mm dishes, washed with phosphate-buffered saline (PBS), and lysed as previously described (9). Proteins were electrophoresed on polyacrylamide gels under reducing conditions, and then blotted onto nitrocellulose filters. Filters were blocked with non-fat dried milk and followed by incubation with a primary Ab against human PEDF, human vascular endothelial growth factor (VEGF) (Santa Cruz Biotechnology, Santa Cruz, Calif.), or α-tubulin (Santa Cruz Biotechnology). After incubation, the filters were treated with horseradish peroxidase-conjugated anti-rabbit IgG (Amersham Pharmacia Biotech, New Jersey), and the resultant immune complexes were visualized using an enhanced chemiluminescence detection system (ECL) (Amersham) as previously described (8). The result is shown in FIG. 1B.

1.5 Results

The human malignant melanoma cell lines G361 and A375 were chosen for their endogenous expression of PEDF and VEGF. As shown in FIGS. 1A and 1B, G361 and A375 cells were found to express substantial amounts of PEDF, and the expression levels of PEDF in these tumor cells were comparable with that of normal human cultured melanocytes. In contrast, expression levels of VEGF among these cells were quite different; G361 and A375 cells were characterized by a strong expression of VEGF, while little VEGF protein was detected in cell lysates from normal melanocytes (FIG. 1B).

1.5 Discussion

The present observations were demonstrated in this study for the first time that the two human malignant melanoma cell lines G361 and A375 expressed substantial amounts of PEDF protein and that the expression levels of PEDF in cell lysates were comparable with that of normal (non-maligant) human melanocytes. This suggests that a decrease or loss in PEDF production alone might not contribute to malignant melanoma development. To elucidate the physiological roles of PEDF derived from normal melanocytes in skin would be beyond the original scope of this study. In this study, we found that two melanoma cell lines express high levels of VEGF proteins, while normal melanocytes produced a minimal amount of VEGF. Melanocytes are transformed to malignant melanoma cells by constitutive signal activation via a mitogen-activated protein kinase, a process that is associated with an increase in production of VEGF, supporting the hypothesis for a functional role of VEGF in melanoma transformation (13).

### Example 1

### Preparations of PEDF-expression vector, and expression vector for purification of PEDF

PEDF cDNA was originally cloned from a human placenta cDNA library (Clontech, Palo Alto, Calif.), and inserted into the mammalian expression vector pBK-CMV (Stratagene, La Jolla, Calif.) as described previously (8).

In brief, the gene encoding PEDF was isolated from the cDNA library by PCR according to the following conditions:

| | |
|---|---|
| PCR Primer | |
| Fw: 5′-CTCAGTGTGCAGGCTTAGAG-3′ | (SEQ ID NO: 4) |
| Rev: 5′-CCTTCGTGTCCTGTGGAATC-3′ | (SEQ ID NO: 5) |
| x 10 pfu buffer | 4 μl |
| dNTP (2 mM each) | 4 μl |
| primer (Fw) (10 μM) | 4 μl |
| primer (Rev) (10 μM) | 4 μl |
| templates (200 mg) | |
| pfu polymerase (STRATAGENE) | 0.5 μl |
| distilled H₂O | /total 40 μl |

Condition of PCR (Parkin Elmer 2400)
(step 1 × 1)

| | |
|---|---|
| 95° C. | 5 min |

(step 2 × 30)

| | |
|---|---|
| 95° C. | 0.5 min |
| 60° C. | 0.5 min |
| 72° C. | 1.5 min |

(step 3 × 1)

| | |
|---|---|
| 72° C. | 10 min |
| 4° C. | ∞min |

After confirmation of the amplification, the PCR product was ligated to the SmaI site of pBluescript II KS. The construction was confirmed by digestion with restriction enzymes and sequencing, and also checked to contain the Xba I site at the 5′ side.

The PEDF PCR product that had been cut with Xba I and Hind III (bulnted) was cloned into PBK-CMV (STRATAGENE) between Nhe I (blunted) and Xba I sites, to give a PEDF-expression vector, plasmid pBK-CMV-PEDF.

Then, an expression vector for purification of PEDF was constructed according to the following procedure.

OligoDNAs for His-Tag, 5′-AATTCCATCATCATCATCATCATTAAT-3′ (SEQ ID NO: 6) and 5′-CTAGATTAATGATGATGATGATGATGG-3′ (SEQ ID NO: 7) were synthesized, and annealed each other. To eliminate the stop codon of PEDF and insert an Eco RI site at the 3′ end, oligoDNA was synthesized by PCR using 5′-CGGAATTCGGGGCCCCTGGGGTCC-3′ (SEQ ID NO: 8) and Fw primer (SEQ ID NO: 4) as described above, and using pBK-CMV-PEDF as template. The amplified product was cut with Bgl II and Eco RI, and the cut product was ligated to the annealed oligoDNA for His Tag as described above. The ligated product was inserted into the purified pBK-CMV-PEDF which had been cut with Bgl II and Xba I. The construction was confirmed by digestion with restriction enzymes and sequencing.

To insert a translational enhancer, the enhancer segment which was isolated from pcDNA4-HisMax (Invitrogen) using Sac I and Xba I, was ligated to the same restriction enzyme sites of pBluescript II KS. After isolation of the clone, the vector was cut at the Nco I site (blunted) and Bam HI site to give the vector backbone.

To modify the 5′ end of PEDF, an oligoDNA was synthesized using 5′-GCATGCAGGCCCTGGTGCTACTCC-3′ (SEQ ID NO: 9) (Sph I site was inserted to the PEDF 5′ end) and 5′-TTAGGTACCATGGATGTCTGGGCT-3′ (SEQ ID NO: 10), and using pBK-CMV-PEDF as template, and the synthesized product was inserted into PGEM-T easy (Promega). The clone was isolated, and then cut at Sph I site (blunted) and Bam HI site. The resulting fragment was inserted into the vector backbone as described above.

The translational enhancer-PEDF5′ portion was removed from the resulting vector by cutting its Sac I site (blunted) and Bam HI site. The fragment containing the translational enhancer-PEDF5′ portion was ligated to pBK-CMV-PEDF having 3′ His-Tag which had been cut at the Nhe I site (blunted) and Bam HI site to give an expression vector for purification of PEDF. The resulting vector was confirmed by digestion with restriction enzyme and sequencing.

### Example 2

### Preparation of PEDF-Overexpressing Transfectants

Subconfluent G361 or A375 cultures were stably transfected either with a PEDF expression vector prepared in Example 1 or with an expression vector pBK-CMV alone using the FuGENE 6 transfection reagent. Twenty-four hours after transfection, cells were split 1:3 into their full growth medium containing 400 μg/ml Zeocin (Invitrogen) to select transfectants. Stably transfected clones were expanded, and the clones were characterized for PEDF production.

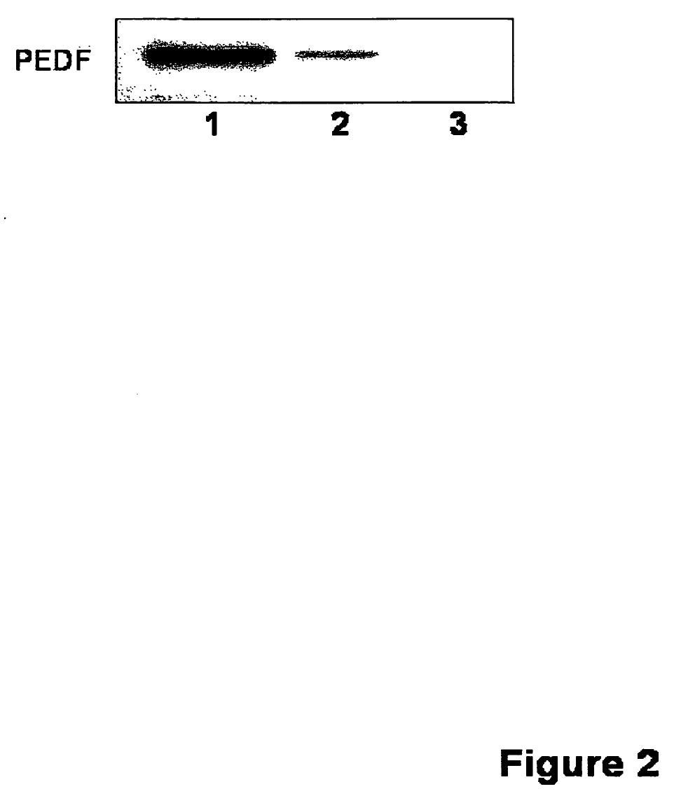

Conditioned medium was obtained from the stably transfected G361 cells grown for 48 h in serum-free culture medium, and then concentrated twenty-fold using Microcon 10 MWCO filters (Amicon, Beverly, Mass.). Proteins were electrophoresed on polyacrylamide gels under reducing conditions, and then blotted onto nitrocellulose filters. Filters were blocked with non-fat dried milk and followed by incubation with a primary Ab against human PEDF. After incubation, the filters were treated with horseradish peroxidase-conjugated anti-rabbit IgG (Amersham Pharmacia Biotech, New Jersey), and the resultant immune complexes were visualized using an enhanced chemiluminescence detection system (ECL) (Amersham) as previously described (8). The result is shown in FIG. 2 wherein lane 1 is the result of purified PEDF proteins used as a positive control, lane 2 is the result of G361 cells stably transfected with the human PEDF expression vector, and lane 3 is the result of G361 cells transfected with the expression vector pBK-CMV alone.

Purified PEDF proteins used as a positive control were prepared as follows. 293T cells (ATCC No. CRL-11268, ATCC, Rockville, Md.) were transfected with an expression vector for purification of PEDF in Example 1 using the FuGENE 6 transfection reagent (Roche Diagnostics, Mannheim, Germany) according to the manufacturer's instructions. Then, PEDF proteins were purified from conditioned media by a Ni-NTA spin kit (Qiagen GmbH, Hilden, Germany) according to the manufacture's instructions. SDS-PAGE analysis of purified PEDF proteins revealed a single band with a molecular weight of about 50 kDa, which showed reactivity with the previously described Ab against human PEDF (8).

As shown in FIG. 2, PEDF-transfected G361 cells secreted increased amounts of PEDF, whereas control vector-transfected cells expressed little or no supernatant PEDF proteins. PEDF-transfected A375 cells showed similar results (data not shown).

### Example 3

### Effect of PEDF Overexpression on Growth of Melanoma Cells in Vivo

In order to examine whether or not PEDF overexpression influences the growth of G361 cells (melanoma cell line) in vivo, G361 xenografts were transplanted into mice.

BALB/c-nu/nu mice were purchased from Japan Clea (Tokyo) and maintained under specific pathogen free conditions. All animal procedures were conducted according to guidelines provided by the Hokkaido University Institutional Animal Care and Use Committee under an approved protocol.

Confluent G361 cells, stably transfected with the human PEDF expression vector prepared in Example 1 or with the expression vector pBK-CMV alone, were trypsinized and resuspended in PBS at a density of 1×10⁷ cells/ml. One million tumor cells were injected intradermally into the flanks of each 6-week-old female athymic nude mice (n=5). The smallest and largest diameters of the G361 tumors were measured after a 5-day interval using a pair of digital calipers, and the G361 tumor volumes were calculated using the following formula:
volume (mm³)=[(smallest diameter)²×largest diameter)]/2 (10).

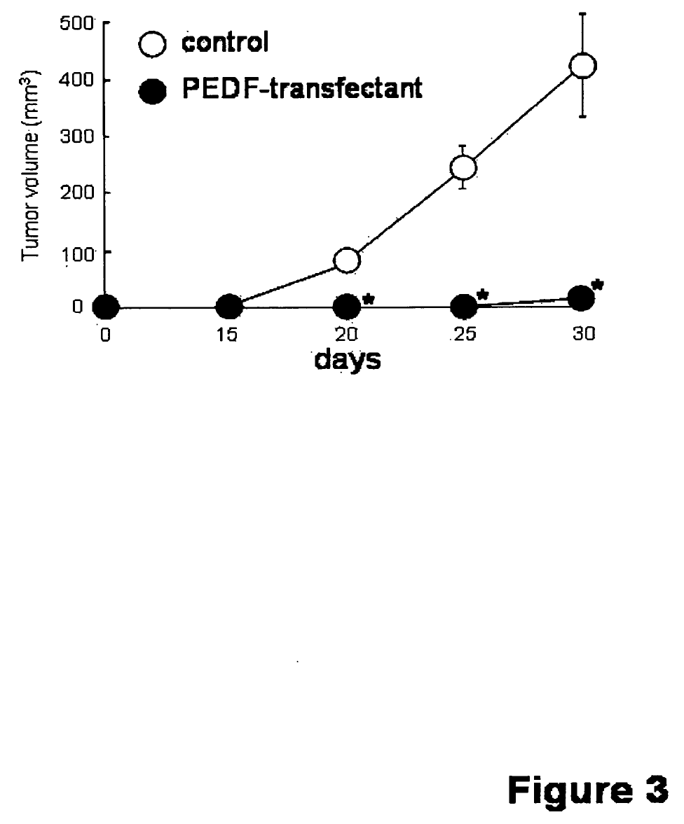

The results are shown in FIG. 3. Control vector-transfected cells formed rapidly growing G361 tumors, reaching 300-400 mm³ after 30 days. In contrast, PEDF overexpression almost completely inhibited in vivo tumor growth of G361 cells over this observation period of up to 30 days.

### Example 4

### Effect of PEDF Overexpression on Angiogenesis in G361 Tumor Mass

To determine the degree of tumor-induced angiogenesis within PEDF-transfected and control vector-transfected G361 tumors, cryostat sections were prepared from G361 tumor xenografts 30 days after implantation. Five different frozen sections of each G361 tumor xenograft were stained with FITC-conjugated rat anti-mouse CD31 (PharMingen, San Diego, Calif.). Nuclei were stained with propidium iodide. Three different fields at 60× magnification were examined on each section using a confocal laser scanning fluorescence microscopy. The percentage of fluorescent-positive areas in 3 different fields from each section was measured.

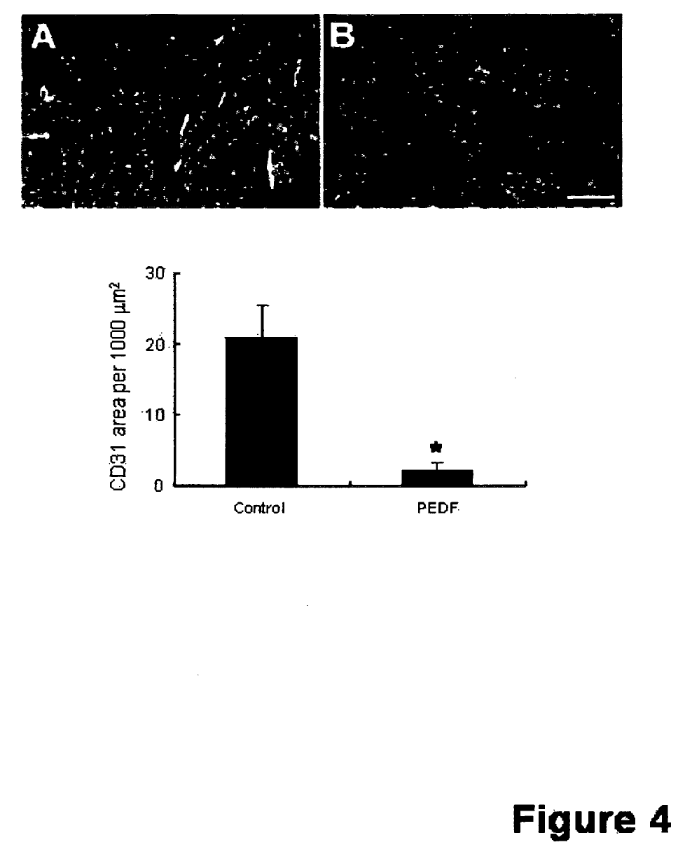

The results are shown in FIG. 4. As shown in FIG. 4, morphometric analysis revealed decreased G361 tumor vessels in 30-day old tumors derived form the PEDF overexpressing G361 cells, compared with control vector-transfected G361 tumor cells.

### Example 5

### Apoptosis of G361 Tumor Cells Caused by PEDF Overexpression

To determine whether PEDF overexpression influences G361 tumor cell proliferation in vitro, growth rates of cultured G361 cells, stably transfected with a human PEDF expression vector or with the expression vector pBK-CMV alone were measured.

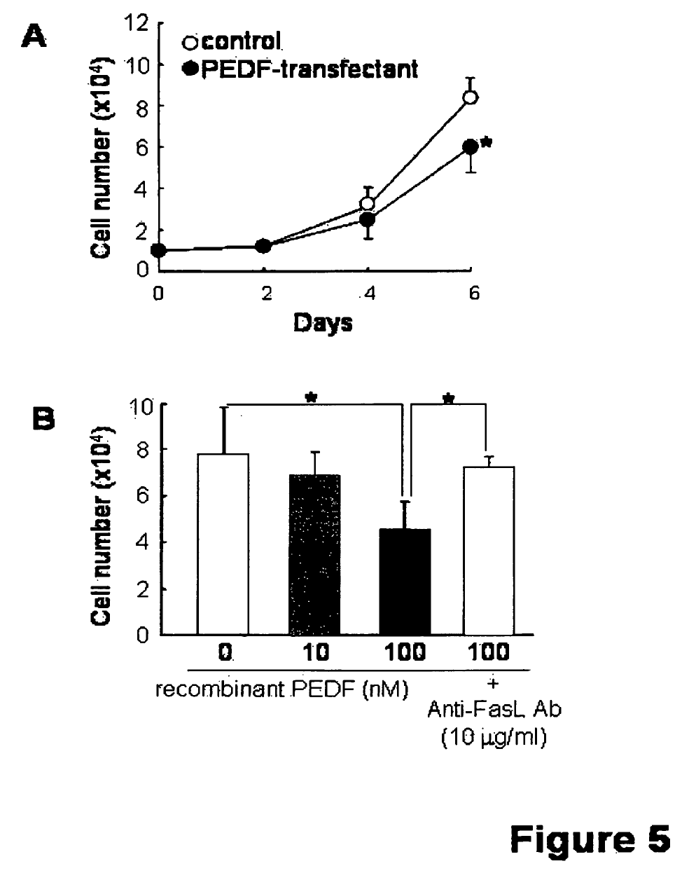

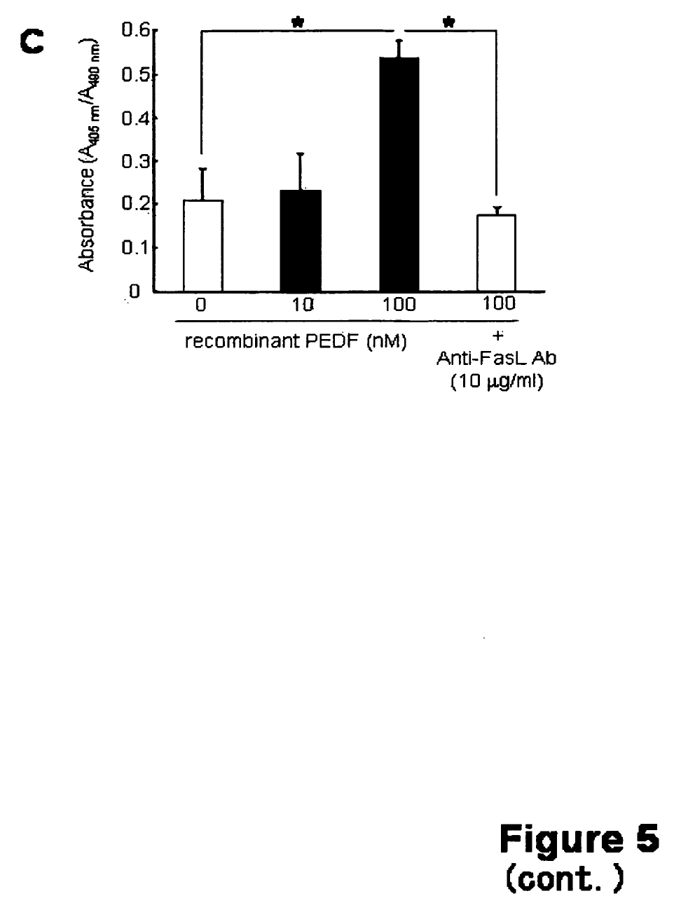

G361 cells, stably transfected with the human PEDF expression vector as prepared in Example 1 or with the expression vector pBK-CMV alone, were serum-starved for 24 h, and cell numbers were counted at days 2, 4, and 6 using a dye exclusion method. The result is shown in FIG. 5A. FIG. 5A shows that growth rates of PEDF transfectants were significantly lower than that of control vector-transfected cells.

Furthermore, in order to investigate effects of PEDF proteins on the growth and apoptosis of non-transfected G361 cells, the cells were incubated with or without various concentrations of PEDF proteins in the presence or absence of 10 μg/ml monoclonal Ab against Fas ligand (PharMingen). After 6-days incubation, viable cell numbers were determined. Apoptosis was measured with an enzyme-linked immunosorbent assay (ELISA) for DNA fragments after 16-h incubation according to the manufacturer's instructions (Cell Death Detection ELISA, Roche Molecular Biochemicals, Mannheim, Germany). PEDF proteins dose-dependently retarded growth and induced apoptotic cell death in cultured non-transfected G361 cells, which was completely blocked by treatments with a neutralizing Ab against Fas ligand (FIGS. 5B and 5C). These results suggest that PEDF elicits apoptosis in G361 cells indirectly, in a Fas ligand-dependent manner.

Then, apoptosis in the PEDF-transfected and control vector-transfected tumors was examined via photomicrography.

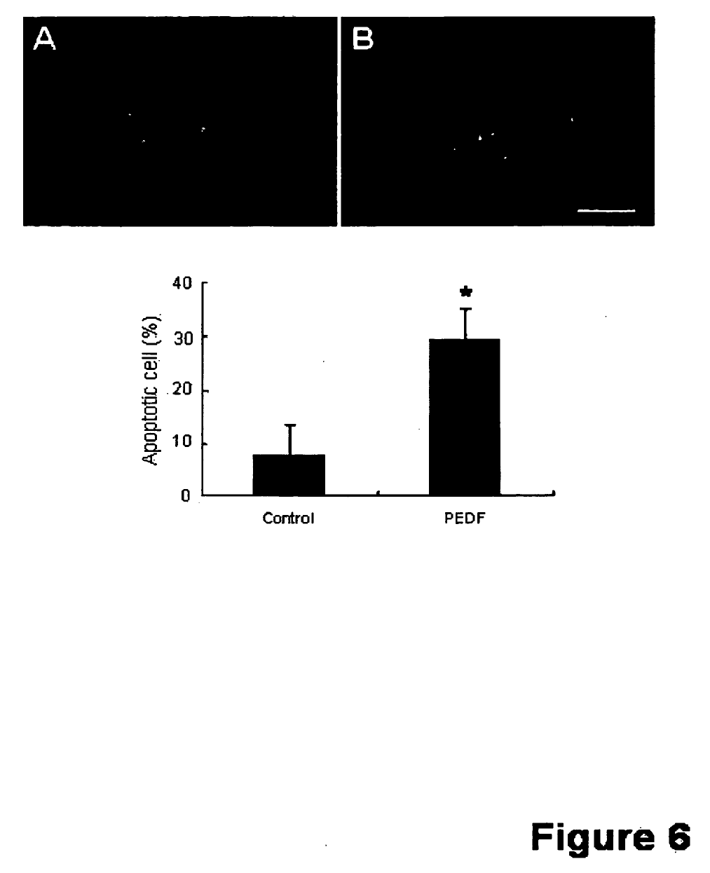

To determine the degree of apoptosis, frozen sections were prepared from G361 tumor xenografts 30 days after implantation. TUNEL assay was performed using in situ apoptosis detection kit (Boeringer Mannheim). The number of apoptotic cells was counted in 10 randomly selected fields at 200× magnification using an immunofluorescence microscopy. The result is shown in FIG. 6. FIG. 6 shows that apoptotic cells were significantly increased in the G361 tumors derived form the PEDF overexpressing G361 cells, compared with control vector-transfected G361 tumor cells.

Effects of the Inventions

Our present study has highlighted two beneficial aspects of the effects of PEDF on melanoma growth and expansion; one is the suppression of melanoma angiogenesis, and the other is induction of Fas ligand-dependent apoptosis in melanoma cells. PEDF is therefore a promising novel therapeutic agent for treatment of patients with melanoma.

### REFERENCES

The contents of the references cited in the specification and below are herein incorporated by reference.
1. Holmgren, L., O'Reilly, M. S., and Folkman, J. Dormancy of micrometastases: balanced proliferation and apoptosis in the presence of angiogenesis suppression. Nat Med. 1:149-53. 1995.
2. Carmeliet, P., and Jain, R. K. Angiogenesis in cancer and other diseases. Nature. 407:249-257. 2000.
3. Scappaticci, F. A. Mechanisms and future directions for angiogenesis-based cancer therapies. J Clin Oncol. 20:3906-3927. 2002.
4. Tombran-Tink, J., Chader, C. G., and Johnson, L. V. PEDF: a pigment epithelium-derived factor with potent neuronal differentiative activity Exp Eye Res. 53:411-414. 1991.
5. Dawson, D. W., Volpert, O. V., Gillis, P., Crawford, S. E., Xu, H. J., Benedict, W., and Bouck, N. P. Pigment epithelium-derived factor: a potent inhibitor of angiogenesis. Science. 285:245-248. 1999.
6. Duh, E. J., Yang, H. S., Suzuma, I., Miyagi, M., Youngman, E., Mori, K., Katai, M., Yan, L., Suzuma, K., West, K., Davarya, S., Tong, P., Gehlbach, P., Pearlman, J., Crabb, J. W., Aiello, L. P., Campochiaro, P. A., and Zack, D. J. Pigment epithelium-derived factor suppresses ischemia-induced retinal neovascularization and VEGF-induced migration and growth. Invest Ophthalmol Vis Sci. 43:821-829, 2002.
7. Spranger, J., Osterhoff, M., Reimann, M., Mohlig, M., Ristow, M., Francis, M. K., Cristofalo, V., Hammes, H. P., Smith, G., Boulton, M., and Pfeiffer, A. F. Loss of the antiangiogenic pigment epithelium-derived factor in patients with angiogenic eye disease. Diabetes. 50:2641-26415. 2001.
8. Yamagishi, S., Inagaki, Y., Amano, S., Okamoto, T., Takeuchi, M., and Makita, Z. Pigment epitheliun-derived factor protects cultured retinal pericytes from advanced glycation end product-induced injury through its antioxidative properties. Biochem Biophys Res Commun. 296:877-882. 2002.
9. Shimizu, T., Abe, R., Nakamura, H., Ohkawara, A., Suzuki, M., and Nishihira, J. High expression of macrophage migration inhibitory factor in human melanoma cells and its role in tumor cell growth and angiogenesis. Biochem Biophys Res Commun. 264:751-758. 1999.
10. Taetle, R., F. Rosen, I. Abramson, J. Venditti, S. Howell. 1987. Use of nude mouse xenografts as preclinical drug screens: in vivo activity of established chemotherapeutic agents against melanoma and ovarian carcinoma xenografts. Cancer Treat Rep. 71:297.
11. Rofstad, E. K., and Halsoer, E. F. Vascular endothelial growth factor, interleukin 8, platelet-derived endothelial cell growth factor, and basic fibroblast growth factor promote angiogenesis and metastasis in human melanoma xenografts. Cancer Res. 60:4932-4938. 2000.
12. Reiher, F. K., Volpert, O. V., Jimenez, B., Crawford, S. E., Dinney, C. P., Henkin, J., Haviv, F., Bouck, N. P., and Campbell, S. C. Inhibition of tumor growth by systemic treatment with thrombospondin-1 peptide mimetics. Int J Cancer. 98:682-689. 2002.
13. Govindarajan, B., Bai, X., Cohen, C., Zhong, H., Kilroy, S., Louis, G., Moses, M., and Arbiser, J. L. Malignant transformation of melanocytes to melanoma by constitutive activation of mitogen-activated protein kinase kinase (MAPKK) signaling. J Biol. Chem. 278:9790-9795. 2003.
14. Holekamp, N. M., Bouck, N., and Volpert, O. Pigment epithelium-derived factor is deficient in the vitreous of patients with choroidal neovascularization due to age-related macular degeneration. Am J Ophthalmol. 134:220227. 2002.
15. Doll, J. A., Stellmach, V. M., Bouck, N. P., Bergh, A. R., Lee, C., Abramson, L. P., Cornwell, M. L., Pins, M. R., Borensztajn, J., and Crawford, S. E. Pigment epithelium-derived factor regulates the vasculature and mass of the prostate and pancreas. Nat Med. 9:774-80. 2003.
16. Volpert, O. V., Zaichuk, T., Zhou, W., Reiher, F., Ferguson, T. A., Stuart, P. M., Amin, M., and Bouck, N. P. Inducer-stimulated Fas targets activated endothelium for destruction by anti-angiogenic thrombospondin-1 and pigment epithelium-derived factor. Nat Med. 8:349-57. 2002.
17. Chen, Y. L., Wang, J. Y., Chen, S. H., and Yang, B. C. Granulocytes mediates the Fas-L-associated apoptosis during lung metastasis of melanoma that determines the metastatic behaviour. Br J Cancer. 87:359-365. 2002.

1. A method for preventing or treating malignant melanoma, which comprises administering an effective amount of at least one selected from the group consisting of:
a) a pigment epithelium-derived factor; and
b) a variant of the pigment epithelium-derived factor (a) that has the functionally equivalent properties to the factor (a)
to a subject in need thereof.

2. The method of claim 1, wherein the Pigment epithelium-derived factor is that derived from human.

3. A method for preventing or treating malignant melanoma, which comprises administering an effective amount of a vector that comprises the nucleic acid molecule encoding at least one selected from the group consisting of
a) a pigment epithelium-derived factor; and
b) a variant of the pigment epithelium-derived factor (a) that has the functionally equivalent properties to the factor (a) to a subject in need thereof.

4. The method of claim 3, wherein the Pigment epithelium-derived factor is that derived from human.


SEQUENCE LISTING (10 sequences)

SEQ ID NO: 1 - protein, 418 aa, homo sapiens [sequence omitted]

SEQ ID NO: 2 - DNA, 1490 bp, homo sapiens
ggacgctggattagaaggcagcaaaaaaagatctgtgctggctggagccccctcagtgtgcaggcttagagggactaggctgggtgtggagctgcagcgtatccacaggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcatgagccccacgaccaacgtgctcctgtctcctctcagtgtggccacggccctctcggccctctcgctgggagcggacgagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctrcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttacgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccct

SEQ ID NO: 3 - protein, 44 aa, Artificial Sequence
  PEDF-derived peptide
VLLSPLSVATALSALSLGAEQRTESIIHRALYYDLISSFDIHGT

SEQ ID NO: 4 - DNA, 20 bp, Artificial Sequence
  PCR primer
ctcagtgtgcaggcttagag

SEQ ID NO: 5 - DNA, 20 bp, Artificial Sequence
  PCR primer
ccttcgtgtcctgtggaatc

SEQ ID NO: 6 - DNA, 27 bp, Artificial Sequence
  OligoDNA for His Tag
aattccatcatcatcatcatcattaat

SEQ ID NO: 7 - DNA, 27 bp, Artificial Sequence
  OligoDNA for His Tag
ctagattaatgatgatgatgatgatgg

SEQ ID NO: 8 - DNA, 24 bp, Artificial Sequence
  PCR primer
cggaattcggggcccctggggtcc

SEQ ID NO: 9 - DNA, 24 bp, Artificial Sequence
  PCR primer
gcatgcaggccctggtgctactcc

SEQ ID NO: 10 - DNA, 24 bp, Artificial Sequence
  PCR primer
ttaggtaccatggatgtctgggct